(19) United States
(12) Patent Application Publication
(10) Pub. No.: US 2018/0125418 A1
(43) Pub. Date: May 10, 2018

(54) DEVICE AND METHOD FOR MONITORING A PHYSIOLOGICAL STATE OF A SUBJECT

(71) Applicant: KONINKLIJKE PHILIPS N.V., EINDHOVEN (NL)

(72) Inventors: Reinder Haakma, Eindhoven (NL); Pedro Miguel Fonseca, Eindhoven (NL); Mustafa Ghassan Radha, Steensel (NL)

(21) Appl. No.: 15/576,421

(22) PCT Filed: May 31, 2016
(86) PCT No.: PCT/EP2016/062201

§ 371 Date: Nov. 22, 2017

(30) Foreign Application Priority Data
Jun. 25, 2015 (EP) 15173834.1

(51) Int. Cl. A61B 5/00; A61B 5/0205; A61B 5/024

(52) U.S. Cl. CPC A61B 5/4812; A61B 5/0205; A61B 5/0245; A61B 5/721; A61B 5/02427; A61B 5/02405

(57) ABSTRACT

The present invention relates to a device (16) and method for monitoring a physiological state of a subject (32). To reduce the energy consumption but still provide a high accuracy, the proposed device comprises a sensor interface (18) for obtaining from a sensor (20) a sensor signal indicative of a vital sign of a subject; a power storage interface (22) for obtaining a charge value indicative of a charge state of a power storage (24) powering the sensor; a duty cycle module (28) for controlling the duty cycle of the sensor based on the charge value; and a processing unit (26) for extracting at least one feature indicative of a physiological state of the subject from the sensor signal.

### FIELD OF THE INVENTION

The present invention relates to a device and method for monitoring a physiological state of a subject as well as to a wearable monitoring apparatus.

### BACKGROUND OF THE INVENTION

Sleep staging is the process of annotating sleep stages of a living being. Usually, physiological recordings of the subject's sleep are examined. The process of sleep staging is standardized by the American Academy of Sleep Medicine (AASM) and described in a corresponding manual. The AASM defines sleep stages on the basis of electroencephalogram (EEG), electromyogram (EMG) and electroocculogram (EOG) measurements, together called a polysomnogram (PSG).

Sleep staging results in a hypnogram, which shows the development of the sleep stages of a subject during a sleep period. The AASM defines sleep stages per 30 second segment of sleep. The hypnogram can be used as an insightful tool in the examination of sleep. The hypnogram is used in clinical settings (e.g. for the diagnosis of sleep disorders), personal settings (e.g. for the user to learn about their sleep patterns), research settings (e.g. to understand relations between sleep patterns and other aspects of a living being's life) as well as other settings.

Recently, a large number of algorithms have been developed that can do sleep staging automatically using a computational model that deduces the sleep stage from PSG data, more specifically from EEG, and optionally also from EMG and EOG (Agarwal, R., & Gotman, J., Computer-assisted sleep staging. Biomedical Engineering, IEEE Transactions on, 48(12), 1412-1423, 2001). The use of automatic algorithms dramatically reduces the amount of labor that is associated with sleep scoring by the trained sleep technician, making sleep staging available to more people.

As the normal procedure described by the AASM for sleep staging requires a set of obtrusive PSG sensors placed on the face and head of the subject, the technique is less suitable for (unsupervised) home monitoring and less attractive for personal use. This raises an interest from both the clinical as well as the personal electronics worlds in unobtrusive methods for sleep staging that can be performed outside a lab and with minimal encumbrance from sensors. Many alternatives for the traditional EEG-based sleep staging have been proposed. One such method that is particularly suitable for unobtrusive sleep staging is based on heart-rate variability (HRV) and body movement. This method requires a sensor that records movements (e.g. an accelerometer) and a sensor that can record the times at which the heart beats (e.g. wearable electrocardiogram, ECG, or photoplethysmogram, PPG). The latter enables the extraction of inter-beat-intervals (IBI) which can then be used to analyze the heart-rate variability (Redmond, S. J., de Chazal, P., O'Brien, C., Ryan, S., McNicholas, W. T., & Heneghan, C., “Sleep staging using cardiorespiratory signals”, Somnologie-Schlafforschung and Schlafmedizin, 11(4), 245-256, 2007). Sleep staging based on HRV is possible due to the dynamics between the sympathetic and parasympathetic components of the autonomous nervous system during different sleep stages, which are reflected in the HRV.

Sleep staging is then typically done in the following way: from the inter-beat-intervals, sleep stage correlated HRV features are extracted (usually over 5 minute windows to account for the slowest IBI variations and these features are used to predict sleep stages (e.g. by using to train a predictive model to separate between sleep stages).

A problem with the use wearable sensors in general and in particular with regard to the monitoring of slowly changing physiological states, e.g. applications such as sleep staging, is the fact that the wearable sensor has to be switched on for a prolonged period of time (e.g. at least one full night) without connection to a power supply. This puts significant demands on the battery, adapter or other mobile power source used to power the sensor. Enlarging the mobile power source is often not an attractive option as it makes the device more cumbersome and uncomfortable for the user. Thus, approaches for power saving are required.

In U.S. Pat. No. 8,945,017 B2 a wearable heart rate monitor is presented. A biometric monitoring device is used to determine a user's heart rate by using a heartbeat waveform sensor and a motion detecting sensor. In some embodiments, the device collects concurrent output data from the heartbeat waveform sensor and output data from the motion detecting sensor, detects a periodic component of the output data from the motion detecting sensor, and uses the periodic component of the output data from the motion detecting sensor to remove a corresponding periodic component from the output data from the heartbeat waveform sensor. From this result, the device may determine and present the user's heart rate.

There is, however, still a need to save energy when providing a longer term monitoring.

### SUMMARY OF THE INVENTION

It is an object of the present invention to provide a monitoring device and method that allow monitoring a physiological state of a subject, which require less energy but provide an accuracy that is sufficient for being used in medical facilities.

In a first aspect of the present invention a device for monitoring a physiological state of a subject is presented, the device comprising

a sensor interface configured to obtain from a sensor a sensor signal indicative of a vital sign of a subject;

a power storage interface configured to obtain a charge value indicative of a charge state of a power storage powering the sensor;

a duty cycle module configured to control the duty cycle of the sensor based on the charge value; and select at least one of a plurality of signal features associated with the sensor signal based on the duty cycle; and control

a processing unit to extract said at least one selected signal feature of the plurality of signal features indicative of a physiological state of the subject from the sensor signal.

In a further aspect of the present invention a method for monitoring a subject is presented, the method comprising the steps of

obtaining from a sensor a sensor signal indicative of a vital sign of a subject;

obtaining a charge value indicative of a charge state of a power storage powering the sensor;

controlling the duty cycle of the sensor based on the charge value;

selecting at least one of a plurality of signal features associated with the sensor signal based on the duty cycle;

controlling (S18) a processing unit to extract said at least one signal feature of the plurality of signal features and

extracting said at least one selected signal feature of the plurality of signal features indicative of a physiological state of the subject from the sensor signal.

In a third aspect of the present invention a device for monitoring a physiological state of a subject is presented, the device comprising

a sensor interface for obtaining from a sensor a sensor signal indicative of a vital sign of a subject, said sensor being operated at a predefined duty cycle;

a processing unit for extracting at least one feature indicative of a physiological state of the subject from the sensor signal; and

a duty cycle module for selecting at least one of a plurality of signal features based on the predefined duty cycle and controlling a processing unit to extract said at least one selected signal feature from the sensor signal.

In yet another aspect of the present invention a wearable monitoring apparatus is presented, the apparatus comprising

a device as disclosed herein;

a sensor for providing a sensor signal; and

a power storage for powering the sensor and for providing a charge value indicative of a current charge state.

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed methods, computer program and medium have similar and/or identical preferred embodiments as the claimed device and as defined in the dependent claims. It is particularly to be understood that the preferred embodiments as defined in the dependent claims are equally applicable to the device according to the third aspect of the present invention and to the monitoring apparatus.

The present invention is based on the idea of making the duty cycle of the sensor dependent on the current state of charge of a power storage unit. In particular, a physiological state may refer to a sleep stage. The present invention aims at providing an efficient monitoring for subjects (in particular patients) of all ages. The invention makes use of duty cycling for scaling down the amount of time that a sensor is switched on during the recording period while maintaining a reliable (and predictable) performance and enabling flexibility in the constraints on the battery capacity. The duty cycling mechanism of the present invention allows reducing the battery use over time by the amount of duty, which can be scaled according to specific needs.

A device of the present invention may be incorporated in the form of a mobile monitoring device, e.g. a wearable device that can be worn during sleep. The device of the present invention is in communication with a sensor via a sensor interface. The sensor provides a signal indicative of a vital sign of the subject. For instance, the sensor interface may be in communication with a heart rate sensor or a respiratory rate sensor etc. It is to be understood that the sensor interface may be in communication with one sensor or with a plurality of sensors.

Furthermore, the device is in communication with a power storage that provides the sensor with power. The device of the present invention obtains a charge value from this power storage. This charge value is indicative of a charge state of the power storage. For instance, a charge value may represent a charge state of a battery in the form of a percentage value or also in the form of an absolute value representing an amount of energy in a suitable unit. Usually, the power storage and the sensor will be housed in a common housing.

The device of the present invention further includes a processing unit which processes the sensor signal and extracts therefrom a feature indicative of the subject's physiological state. As used herein, a physiological state may, e.g., refer to a sleep stage, an apnea/hypopnea index (AHI), a long-term stress level or to another health-related parameter. The processing unit provides the actual monitoring of the physiological state of the subject.

Initially, the processing unit extracts one or more features from the sensor signal. As used herein, a feature refers to any parameter that can be obtained from evaluating a signal and thus is also termed as a signal feature. For instance, a signal feature can be a maximum or minimum value of the sensor signal during a specified time period. To further elaborate signal feature is a well-known term in the domain of signal processing. Typically, various signal features are extracted from a signal for evaluating the signal. For instance, in the domain of bio-medical signal, such as ECG signal, signal features are extracted using several methods, such as, Time Analysis, Frequency Analysis, Time-Frequency Analysis and Time-Frequency-Space Analysis. Feature extraction leads to specific information from ECG signals, such as peak frequency using time frequency analysis, average power of the ECG signal in a time window. Usually, the processing unit will be configured to determine the current physiological state. For this, the extracted feature or feature set to monitor the development of the physiological state over time is determined. Then, an algorithm (e.g. a regression algorithm, in particular a linear regression algorithm) may be applied to assign a physiological state depending on a value of a feature. This may be particularly useful if a physiological state that can be expressed numerically is to be monitored (e.g. the apnea/hypopnea index or a stress level that can be expressed in the form of a percentage or absolute value). Also, it may be possible to apply a classifier approach. This may be particularly useful if a physiological state that is expressed in categorical terms is to be monitored (e.g. a sleep stage that is expressed in the form of a stage on a predefined scale).

Thus, a feature may refer to an input to an algorithm or classifier for estimating the physiological state. A feature may, however, also refer to a result, i.e., be descriptive of the physiological effect. In other words, a feature may also be determined by using an algorithm or classifier, i.e., a feature may also define a certain algorithm or classifier to be applied.

Furthermore, the device includes a duty cycle module. It is possible that the processing unit and the duty cycle module are incorporated into a single unit, e.g. a microprocessor. The duty cycle module adjusts the duty cycle of the sensor based on the charge value of the power storage.

As used herein, a duty cycle refers to a fraction of time in which a sensor provides a sensor signal. Usually, it is possible to switch the sensor on and off. Thereby, the duty cycle can be controlled. Also, it may be possible to control the duty cycle of the sensor by putting the sensor in a sleep mode or even more generally by modifying a sensor setting that has an effect on the amount of power the sensor consumes (e.g. a power management setting of the sensor).

The duty cycle module executes such a control. The duty cycle indicates how frequently the sensor is powered on and provides a sensor signal. As an example, a pulse duration may correspond to a time the sensor is switched on and a period may correspond to a periodically recurring time period that depends on the physiological state to be monitored. The duty cycle corresponds to the relation of the two parameters. For instance, a sensor may provide a signal for 30 s every two minutes, which corresponds to a duty cycle of 0.25. The duty cycle module may executes the control by considering the amount of power the sensor consumes depending on a current setting on the sensor (e.g. being switched on or off, being put into a sleep mode or not, being configured in a way to save provide a signal with higher noise but consume less power, etc.).

The duty cycle module thus allows controlling the amount of power being consumed since a lower duty cycle usually results in a smaller amount of energy being consumed. If the duty cycle is lower this means that the sensor is not powered on all the time. In times when the sensor is not powered on the sensor does not consume power. Thus, power can be saved. This is of particular relevance in mobile devices, since the amount of available energy is usually a limiting factor for the maximum operating time of the device. The present invention may allow operating a mobile device for a longer time.

Also, the required energy is a factor when it comes to designing a housing for housing a mobile power storage such as a battery. A larger battery requires more space and is more expensive. The present invention may result in a smaller battery size and in reduced costs.

In comparison to previous approaches to duty cycling the present invention directly exploits the available amount of energy. If only a small amount of energy is left, the duty cycle can be reduced to reduce energy consumption and thereby increase the lifetime.

The duty cycle module is configured to select at least one of a plurality of signal features, preferably two, based on the duty cycle; and control the processing unit to extract said at least one selected signal feature from the sensor signal. The duty cycle also has an effect on the feature selection. Depending on the physiological state of interest, it is usually possible to monitor a plurality of different signal features that are all indicative to a higher or lower degree of the physiological state. However, different of these signal features may be differently affected by a varying duty cycle. One feature may, e.g., be of high significance if a signal of a high duty cycle is available but may be very susceptible to a reduction of the duty cycle. In other words, this second signal feature may not be suitable for monitoring the vital state of the subject if the sensor signal is not available at a certain minimum duty cycle.

This relationship is exploited by the duty cycle module in that at least one of the possible signal features is selected based on the determined duty cycle. In other words, the current duty cycle is considered when selecting the feature or feature set for being used in the physiological state monitoring. For instance, it may be possible to make sure that a signal that is only susceptible to a very small degree to a reduced duty cycle is used when the battery consumption, i.e. a charge state of the power storage, indicates that only a small amount of energy is left so that the sensor can only be powered at a lower duty cycle. The duty cycle module controls the processing unit to extract this at least one selected feature. An advantage of assuring that only suitable signal features are selected for a given duty cycle is that it becomes possible to retain an adequate monitoring in spite of a reduced duty cycle. It is assured that always an adequate choice of signal features is monitored.

In another embodiment, the duty cycle is a pre-defined duty cycle. Thus, it is also possible that the sensor interface obtains a sensor signal at the predefined duty cycle. This may, e.g., be the case if different sensors can be connected to a sensor interface. Then, the duty cycle may provide a selection of features to be extracted from this sensor signal being available at a given duty cycle that provides a highest possible significance.

In a preferred embodiment the processing unit is configured to extract at least one feature indicative of a sleep stage of the subject. The present invention is particularly useful when monitoring slowly changing phenomena. The sleep stage of a subject, e.g., changes at comparatively low frequencies in comparison to other body functions that vary at a higher frequency. While a subject is asleep the sleep stage is usually only changed every few minutes or at lower frequencies. Thus, it is also possible to monitor at the sleep stages by means of a sensor signal being sampled at a lower duty cycle. It is, however, to be understood that the present invention may also be used for other physiological phenomena. The invention relates to any wearable technology in which power supply is limited and recording times can be long.

In a preferred embodiment the processing unit is configured to determine a hypnogram of the subject indicative of the sleep stages of the subject during a predefined time period based on said at least one extracted feature. As used herein, a hypnogram refers to a standard visualization of sleep stages of a subject. The different sleep stages are indicated on the y-axis, the time is indicated on the x-axis. The hypnogram allows a treating physician to obtain a quick overview of the sleep cycle of the subject.

In an embodiment, the processing unit is configured to determine the hypnogram based on a classifier with pre-trained parameters, in particular a Bayesian linear discriminant, trained to assign one of a set of predefined sleep stages to a fraction of said predefined time period. It is possible to make use of a classifier, i.e. a statistical approach relying on a data analysis of previously collected data to provide an explanation of data being collected in the future. The use of a classifier allows assigning a sleep stage based on a feature or a feature set. Usually, the parameters of the classifier will be adapted to the respective feature or feature set. Usually, there will be several sets of predefined parameters for the classification and the processing unit will be configured to select the appropriate set depending on the chosen duty cycle and the corresponding set of features measured at that duty cycle. The parameters may also be adapted to a current power management. A feature or feature set is related to a specific sleep stage. Thus, a simple relationship between features and sleep stages can be established. Usually, data that have been collected by means of a more elaborate sleep staging approach are used in the training phase. For instance, such training data may be collected in the sleep laboratory by means of a plurality of on-body sensors, i.e. based on a polysomnogram. Preferably, a Bayesian linear discriminant may be used in which a linear discriminant function is evaluated. This linear function is derived via the Bayes theorem.

In another embodiment the duty cycle module is configured to reduce the duty cycle if the charge value indicates a charge state below a predefined threshold. One preferred control approach includes a reduction of the duty cycle in a situation where the charge value indicates that not much power is available. As soon as the charge state falls below a predefined threshold, the duty cycle is reduced. Such a predefined threshold may be determined from a theoretical analysis of the available amount of power in a power storage.

In a preferred embodiment the duty cycle module is configured to adjust the duty cycle based on at least one of a required operating time of the sensor, a predicted operating time of the sensor and a required accuracy level of a physiological state monitoring. Preferably, a calculation may be undergone that allows determining a duty cycle that allows providing a monitoring for a certain required operating time. For instance, it may be possible to assure that the duty cycle is calculated such that the monitoring can be assured for the rest of the night or for the rest of the week etc. (required operating time). By dynamically adjusting the duty cycle based on a such required operating time it becomes possible to provide an optimized monitoring for a given charge status of a power storage. Alternatively or additionally it may also be possible to make use of a predicted operating time. For instance, if it is known that a monitoring is usually required for two days per week the predicted operating time may correspond to a total operating time until the device or, more precisely, the power storage, has to be recharged. The monitoring may thus be optimized for a given charge state in view of a time the sensor is required to be operable or (required operating time) or in view of the time the sensor is assumed to be operated (predicted operating time). Both the required operating time and the predicted operating time may be determined based on a manual input of a subject or physician or may be calculated based on previous monitoring periods. Furthermore, it may also be possible to adjust the duty cycle based on a required accuracy level of a physiological state monitoring. In other words, this accuracy corresponds to a measure for how well a physiological state being determined based on an extracted feature corresponds to the actual state (i.e. agreement between the classifier's outcome and the truth). Depending on the feature or feature set the resulting accuracy may be low so that too little information for the classifier to determine the sleep stage may be left. In spite of a lower power usage a minimally required accuracy level should be maintained. This minimally required threshold may correspond to a predefined threshold. Such a threshold may, e.g. be defined by a physician or based on study and may be included into an accuracy look-up table.

In a preferred embodiment the sensor interface is configured to obtain at least one of a sensor signal indicative of a heart rate of the subject, in particular an electrocardiography signal and/or a photoplethysmography signal; and a sensor signal indicative of a respiration of the subject, in particular an acceleration signal and/or a photoplethysmography signal. Both, the heart rate and the respiratory rate are known to include information on the sleep stage of a subject. Usually, the heart of a subject beats very regularly and rather slow during a deep sleep period. Also, the breathing is calm. By making use of heart rate or respiratory rate sensors, it becomes possible to exploit this relationship. It may, e.g. be possible to extract a value of a spectral peak in a frequency band from 0.15 to 0.4 Hz of a mean respiratory frequency from a sensor signal indicative of a respiration of the subject.

In a preferred embodiment the sensor interface is configured to obtain a sensor signal indicative of a heart rate of the subject, in particular a photoplethysmography signal, and the duty cycle module is configured to control the processing unit to extract a feature indicative of a heart rate variability, in particular to extract at least one of:

a mean inter beat interval of the heart rate;

a standard deviation of the mean inter beat interval;

a low frequency power parameter indicative of a power in the spectral band between 0.04 and 0.15 Hz;

a high frequency power parameter indicative of a power in the spectral band between 0.15 and 0.4 Hz;

a mean of absolute successive inter beat intervals differences;

a root-mean-square of successive inter beat intervals differences;

a percentage of successive inter beat interval differences larger than 50 ms;

a standard deviation of successive inter beat interval differences;

a phase of the high frequency pole;

a sample entropy; and

a Teager-Kaiser energy.

In particular, the heart rate variability (HRV) is of interest when evaluating a sleep stage of a subject. This HRV can be assessed via different parameters. The HRV describes how the beats of the heart of the subject occur. In particular, the HRV is indicative of the regularity or the variation in the heart beats or, more precisely, the time intervals between the heartbeats (inter beat interval, IBI). The different features are differently susceptible to changes in the duty cycle. Thus, depending on which duty cycle is selected, it may make sense to select one or another feature. Also, it is possible to jointly analyze a set of features. For instance by making use of combined measures being based on an occurrence of certain feature combinations (feature set).

In a preferred embodiment a photoplethysmography (PPG) sensor is used for the monitoring. PPG is very attractive for sleep staging as it can be measured on distal limbs, making it possible to mount the PPG sensor on the lower arm or lower leg using a simple band. Furthermore, no adhesives or contact gels are required (in contrast to ECG), making it a more user-friendly option for personal use. The main limitation of PPG, which is its sensitivity to body movements, is also minimal during sleep as the body is lying still most of the time. In this way, close approximations of the inter-beat-intervals can be derived from the PPG. A PPG sensor provides a sensor signal being indicative of a heart rate and a respiration rate.

In another embodiment the duty cycle module is configured to select the at least one of a plurality of signal features based on a predetermined look-up table indicating for a duty cycle a significance of a feature or feature set with respect to a physiological state of the subject. One option for selecting one or more signal features to be extracted for a certain duty cycle is to make use of a look-up table. In this look-up table, it may be indicated how significant the respective features or feature sets are when predicting/determining a sleep stage (or another physiological phenomenon) if a sensor signal of a specific duty cycle is used. As explained before, different features or feature sets may be differently susceptible to a varying duty cycle. This susceptibility may be measured in a calibration study or in a theoretical analysis in which data sets are artificially transformed into a lower duty cycle by downsampling. In other words, a signal that is recorded during a given time period may form the basis for analyzing the connection between the duty cycle and the significance with respect to a physiological state of a subject when parts of the sensor signal are deleted, i.e. when the analysis is restricted to parts of the sensor signal. Thereby, it becomes possible to derive a duty cycle that indicates how significant the feature, or, more precisely, the determination of the physiological state based on the feature, is for each duty cycle. In a preferred embodiment the look-up table indicates a Cohen's kappa coefficient for a duty cycle and a feature or feature set. Cohen's kappa coefficient is a statistical measure for the inter-rater agreement of multiple features. Cohen's kappa coefficient particularly compensates for an expected random correspondence. Making use of Cohen's kappa coefficient allows obtaining a robust measure for the explanatory power of a feature with respect to a physiological state of the subject for a given duty cycle.

### BRIEF DESCRIPTION OF THE DRAWINGS

These and other aspects of the invention will be apparent from and elucidated with reference to the embodiment(s) described hereinafter. In the following drawings

FIG. 1 shows a sample hypnogram;

FIG. 2 shows a sample for a simultaneous ECG and PPG recording corresponding to a sensor signal being indicative of a heart rate of a subject;

FIG. 3 illustrates the concept of duty cycling;

FIG. 4 illustrates schematically an embodiment of a device and apparatus according to aspects of the present invention;

FIG. 5 schematically illustrates the effect of duty cycling on a few HRV features by way of example;

FIG. 6 schematically illustrates an application scenario of an apparatus according to an aspect of the present invention in the field of sleep staging of a patient; and

FIG. 7 schematically illustrates a method according to the present invention.

### DETAILED DESCRIPTION OF THE INVENTION

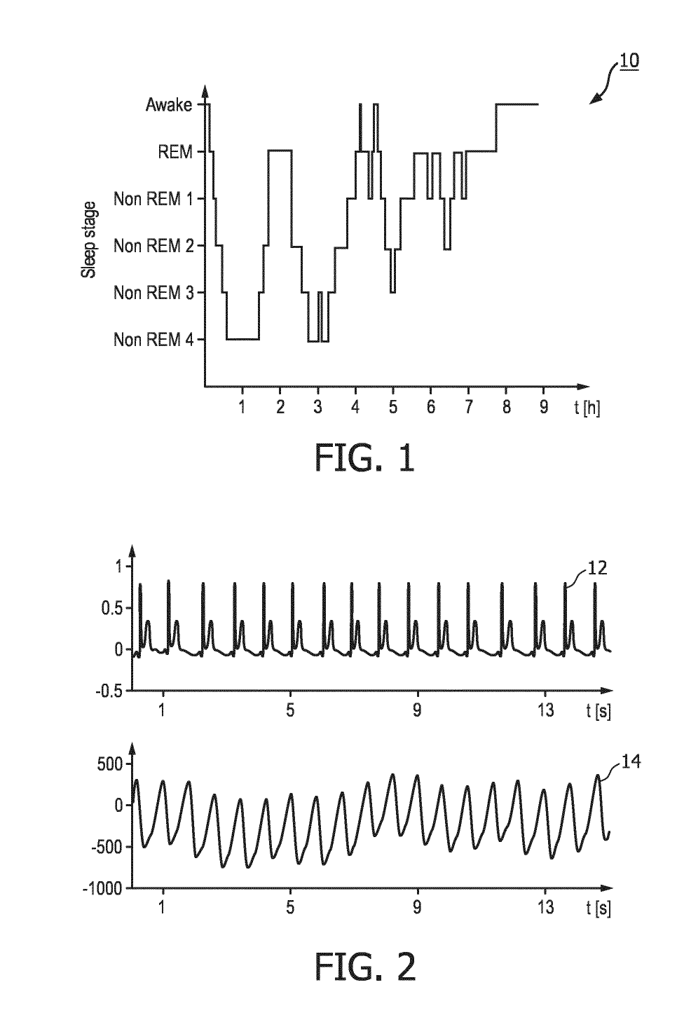

In FIG. 1 an example for a hypnogram 10 is shown. On the x-axis the time during a sleep period of a subject is indicated. On the y-axis each time slot is attributed one of a plurality of different sleep stages. In the illustrated example the sleep stages “Awake”, “REM” and “Non REM 1”-“non REM 4” are used. It is, however, also possible that other scales are used.

The present invention may be put to use in the field of sleep staging for providing a hypnogram of a patient by monitoring one or more of his vital signs. The present invention may, however, also be used for other physiological states such as any condition or state of the body or bodily function. The concept of the present invention is particularly useful for monitoring comparatively slowly changing phenomena that can also be monitored at a lower duty cycle such as an apnea/hypopnea index, a stress level, a body temperature, a level of a drug in the blood of a patient, a mental state, a blood glucose level, a blood pressure, an arterial distensibility, a blood oxygenation, a calorie-expenditure, a sleep quality, a kidney function, a hydration level, a respiratory rate, a cardiac output, a frequency of arrhythmia events, etc. The device or apparatus of the present invention may be incorporated in the form of a device being included in a smartphone or a body-worn device such as a smart watch, a wristband or a heart rate belt etc.

FIG. 2 shows an example for simultaneous electrocardiogram 12 (ECG, top) and photoplethysmogram 14 (PPG, bottom) recordings for a subject. The x-axis is indicative of the time. The concept of the present invention may particularly be exploited for a sensor signal being indicative of a heart rate of a subject such as the illustrated ECG 12 and PPG 14 signals. The correspondence of ECGR-peaks (which denote heartbeats) and PPG pulse peaks is visible. Preferably, a PPG sensor and signal is used.

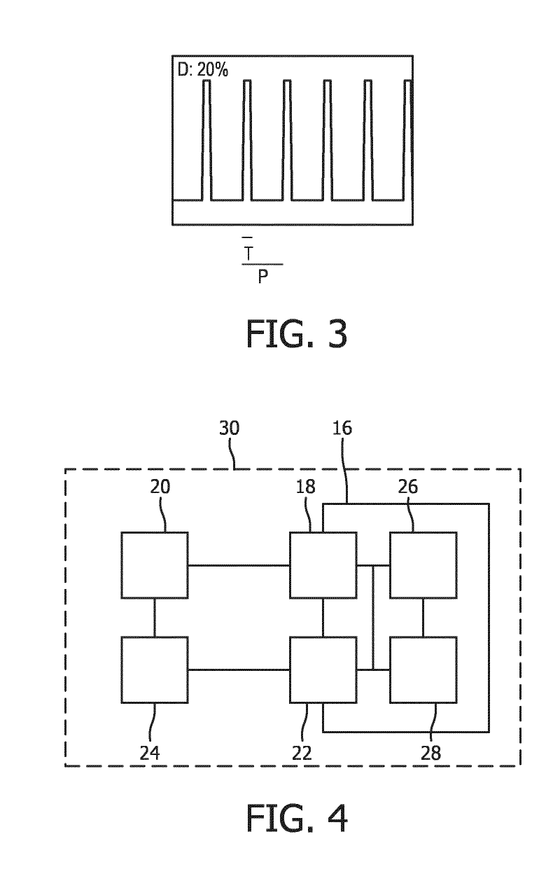

The present invention exploits the concept of duty cycling as illustrated in FIG. 3. A duty cycle is usually defined by a pulse duration T and a period P. The duty cycle D then corresponds to the fraction T/P. In particular, the duty cycle may correspond to the fraction of time a battery is used and the sensor is powered on, i.e. recording a signal. As used herein, duty cycling may particularly correspond to switching the sensor on and off or to putting the sensor into a sleep mode for saving energy.

Both, the duration and the period may be changed. In an embodiment the duty cycle module is configured to adjust the duty cycle by determining a pulse duration for a predefined time interval. The period P may be fixed due to constraints implied by the measurement procedure. Thus, only the pulse duration T may be adjusted by the duty cycle module. When monitoring a physiological state of a subject, the period P usually has an upper bound determined by a size of a time window required for computing a signal feature. The pulse duration T usually has a lower bound determined by a minimal percentage of data required for computing a respective feature with sufficient reliability and significance.

In the context of the present invention the period P may, e.g., be set to 60 s (i.e. the period having a periodicity of 1 minute). The pulse duration T may then have any value between 60 s (duty cycle=60/60=100%), to, e.g., 30 s (duty cycle=30/60=50%). If a pulse duration shorter than 30 s or period shorter period than 60 s is used the reliability and significance of most features that can be extracted from a vital sign signal and that have an explanatory power with respect to sleep staging has been found to drop considerably.

It is, however, to be understood that other physiological states to be monitored may allow making use of other values for P and T.

In the example illustrated in FIG. 3 the period P is about five times as long as the pulse duration T leading to a duty cycle D of 20%.

FIG. 4 schematically illustrates a device 16 for monitoring a physiological state of a subject according to an aspect of the present invention. The device 16 corresponds to a wearable heart-rate variability based unobtrusive sleep staging system including a PPG sensor and an acceleration sensor that is augmented with a duty cycling mechanic that allows for scalable battery consumption for the device with a predictable and graceful performance degradation when battery consumption is scaled down.

The device 16 allows reducing the battery consumption of the sensor dramatically (e.g. up to a half) while still maintaining a predictable performance that degrades gracefully as the duty cycle is decreased. The invention is particularly interesting for the analysis of heart-rate variability and other inter-beat-interval features from a PPG sensor that will be commercialized for personal use in daily life and thus has to be able to handle long periods of usage without maintenance (i.e. recharging of the battery) while still keeping the size of the battery minimal to ensure a comfortable and unobtrusive user experience.

The device 16 includes a sensor interface 18 which is in communication with a sensor 20 and a power storage interface 22 which is in communication with a power storage 24. The device 16 further includes a processing unit 26 which processes the sensor signal from the sensor 20. Still further, the device 16 includes a duty cycle module 28 for controlling a duty cycle of the sensor 20. Some or all of the interfaces, units and modules of the device of the present invention may be partly or completely implemented in hard- and/or software. It may be possible that some of all functions are provided by a single microprocessor unit.

The device 16 may, e.g., be represented by a handheld device being in communication with a wearable sensor. It may, however, also be possible that the device 16 is represented by an online server being in communication with a device including a sensor and the power storage and being applied to the subject and communicating with said on-body device by a network or internet connection.

In an aspect of the present invention the device 16, the sensor 20 and the power storage 24 may be incorporated into an apparatus 30 being designed as a wearable monitoring apparatus to be used for sleep monitoring.

In a preferred embodiment the device of the present invention is used for monitoring a sleep stage or sleep state of a patient by determining a hypnogram being indicative of the sleep stages of the patient. The actual sleep monitoring as provided by the processing unit 26 is usually based on feature extraction from a sensor signal. One or more features can be extracted and used for assigning a sleep stage to a current time period. Preferably, a set of cardiac features is extracted from a sensor signal that is provided by a sensor being applied to the subject. For instance, an ECG or PPG sensor can be used. In a preferred embodiment, a PPG sensor is used that provides a PPG signal. The PPG signal is indicative of the heart rate and respiratory rate of the subject.

In another preferred embodiment an acceleration sensor attached to the subjects chest (e.g. in a chest belt) is used in addition to the PPG sensor for providing an acceleration signal indicative of the respiration of the subject. In other embodiments, however, it is possible that further sensors are used in addition to the vital sign sensor such as a skin temperature sensor, an ambient temperature sensor, an ambient light level sensor or a galvanic skin response sensor.

In particular, the processing device exploits that some cardiac features can be extracted from a sensor without losing too much information even if the sensor signal is only available at a reduced duty cycle.

The duty cycle module 28 controls the sensor 20. This control may, e.g., be applied via the sensor interface 18. In particular, the duty cycle module 28 controls whether or not the sensor is switched on, i.e. is on duty and consumes energy (or consumes a higher amount of energy than while being in a sleep mode upon availability).

The processing unit 26 may be configured to apply a sleep monitoring algorithm which uses the extracted cardiac features to automatically determine one or more sleep parameters that can then be used to derive a hypnogram.

In a preferred embodiment a sleep monitoring algorithm may include the use of a Bayesian linear discriminant which is chained to classify each epoch of a sleep phase of a patient into one of the sleep stages “wake”, “light sleep”, “deep sleep”, and “REM”. However, other classifiers may perform comparably well. Also, it is possible to use other sleep stage labels such as: “wake”, “non-REM”, “REM”.

In the application area of sleep staging the extracted features may particularly correspond to well-known heart rate variability (HRV) parameters that are established to be discriminative of different sleep stages.

For each one-minute period during a sleep period of a patient, a HRV window is defined that encompasses the two one-minute periods prior and the two one-minute periods posterior to the current period. In each of these one-minute periods the sensor is switched on for a pulse duration of 30 s (corresponding to a duty cycle of 0.5). This five-minute-window is extracted from the sensor signal provided by the sensor 20. The core idea is that for each instance to be classified as a sleep stage there will be a window centered on it over which the HRV features will be computed.

It is to be understood that also other times can be used. The length of the window is of lesser importance, but a period of approximately 5 minutes is often used in order to identify the very low frequency components. These very low frequencies (VLF) can be as low as 0.003 Hz, which means that a full oscillation would take 1/0.003=333 seconds˜5.5 minutes. In another embodiment, it may, e.g., be possible that for each 30 s interval during a sleep period of a patient, a HRV window is defined that encompasses the two minutes prior to the current interval, the current interval itself and the two minutes posterior to the current interval.

For sleep staging, particularly the inter-beat intervals (IBI) are evaluated to extract HRV features. Common cardiac features that are used for sleep staging and that may be extracted by the processing unit 26 of the present invention include: the mean of the IBIs (mean NN), the standard deviation of the IBIs (SDNN), the power in the spectral band between 0.04 and 0.15 Hz (low frequency), the power in the spectral band between 0.15 and 0.4 Hz (high frequency), the mean of absolute successive differences (MAD), the route-mean-square of successive differences (RMSSD), the percentage of successive IBI differences larger than 50 ms (PNN50), the standard deviation of successive IBI differences (SDSD), a value representing a spectral peak in the high frequency band from 0.15 to 0.4 Hz and corresponding to a power of the mean respiratory frequency (the respiratory frequency can also be extracted from a PPG signal in addition to features being indicative of the heart rate), the phase of the heart frequency pole (cf. Mendez, M., Bianchi, A. M., Villantieri, O., & Cerutti, S., “Time-varying analysis of the heart rate variability during REM and non REM sleep stages”, Engineering in Medicine and Biology Society, EMBS′06 28th Annual International Conference of the IEEE, 2006), the sample entropy (cf. Costa, M., Goldberger, A. L., & Peng, C. K., “Multiscale entropy analysis of biological signals”, Physical Review E, 71(2), 021906, 2005) and the Teager-Kaiser, i.e. the Teager energy and the Teager size (cf. Eivind Kvedalen, “Signal processing using the Teager Energy Operator and other nonlinear operators”, May 2003 or Chandrakar Kamath, “A new approach to detect congestive heart failure using Teager energy nonlinear scatter plot of R-R interval series”, Medical Engineering and Physics, 2012, Volume 34, Issue 7, Pages 841-848). Based on the Teager-Kaiser energy it is also possible to determine a Teager energy and/or a Teager size. As used herein, the Teager-Kaiser energy is computed for each heart-beat in a specific window of heart beats. The Teager energy relates to the average Teager-Kaiser energy of all beats in a window. The Teager size relates to the proportion of beats in the window that have a Teager energy above a pre-defined threshold.

It is to be understood that also other features may be used in a context of sleep staging and when monitoring other physiological states of a subject.

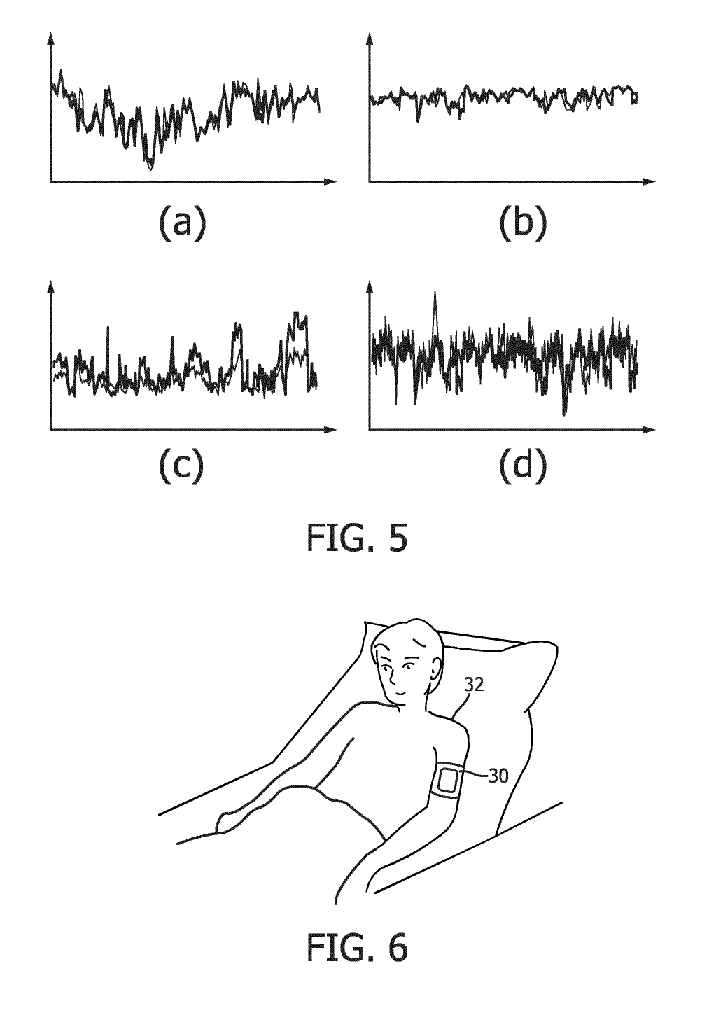

FIG. 5 schematically illustrates for a number of the above-outlined features how they are affected when the duty cycle is scaled down from 100% to 50%. The thick line denotes the respective feature at a duty cycle of 100%. The thin line denotes the respective feature at a duty cycle of 50%. In FIG. 4A the root mean square of successive IBI differences (RMSSD) is illustrated, in FIG. 4B the sample entropy at a scale of 1 is illustrated, in FIG. 5C the Teager-Kaiser energy is illustrated (i.e. a percentage of R-R intervals in a window that have a Teager-Kaiser energy above a predefined threshold, which may also be referred to as Teager size) and in FIG. 5D the sample entropy at a scale of 10 is illustrated.

FIG. 5 shows that the dynamics of the respective features are largely retained in spite of the reduced duty cycle. All of the HRV features retain a Pearson's correlation of at least 0.8 when extracted based on a duty cycle of 50% with respect to their respective counterparts extracted based on a duty cycle of 100% on a data set comprising 26 PPG night-sleep recordings.

This example shows that it is still possible to reliably determine a sleep stage even if the duty cycle of the sensor is reduced.

An additional effect of the duty cycling scheme of the present invention is that the resulting sleep staging performance becomes predictable to a certain degree. A performance of an IBI-based automatic sleep stage classifier may be evaluated based on Cohen's kappa, a measure of agreement factoring in agreement by chance. The following table shows the performance of an IBI-based automatic sleep stage classifier for a data set of 26 night time sleep recordings. In particular, the table shows the performance in the form of Cohen's kappa coefficient for different duty cycles with P=60 s.

| Duty | | |
|---|---|---|
| cycle | Feature set | kappa |
| 100% | All features | 0.48 |
| 100% | Robust subset | 0.45 |
| 75% | All features | 0.46 |
| 50% | All features | 0.42 |
| 50% | Robust subset | 0.43 |

Cohen's kappa coefficient is the proportion of observed agreement (P₀) to hypothetical agreement (Pₑ) and is calculated as K=(P₀−Pₑ)/1−Pₑ. It can be seen in the table that the performance for a larger set of HRV features does not maintain a high correlation for a reduced duty cycle.

In an example, a very robust subset can be extracted from 50% duty cycled data and includes: a SDNN, a SDSD, an HF, a mean HR, percentiles, a Teager-Kaiser energy (and size), a phase coordination. The robust subset may work with 75% and include an LF, a LF/HF and a VLF in addition to the above features of the very robust subset. A full set may work at 100% and include a likelihood of cognitive arousals, a sample entropy and auto-regressive coefficients in addition to all of the above. The full set of features yields a lower Cohen's kappa than the robust subset when computed on 50% duty cycling. The robust subset of features yields a lower Cohen's kappa than the full set when computed a 100% duty cycle. Thus, it can be seen that the subset of features being robust to duty cycling outperforms the full set of features at a duty cycle of 50%. Furthermore, it can be seen that the performance decreases gracefully with the reduction of the duty cycle.

The duty cycle module may thus provide an automatic determination and adjustment of P and T based on the current context. For example, an available battery life at the beginning of a night may be used for determining a duty cycle of the sensor for the entire night (required operating time). In other words, P and T may be determined such that the available battery life is sufficient for powering the sensor throughout the entire remaining night. Another option is that the duty cycle module determines and adjusts P and T in anticipation of how many nights the device should be able to record before the battery must be recharged (predicted operating time).

The selection of features to be extracted by the processing unit can thus be made dependent on whether the features still provide reliable enough information to perform sleep monitoring for a given duty cycle. If the duty cycle is adjusted the determination of features to extract will also be adjusted frequently. For instance, it may be possible to make use of a look-up table corresponding to a predefined list of features which are known beforehand to handle different duty cycles without loss in sleep monitoring accuracy or performance. In other words, in a calibration procedure or in an evaluation procedure it may be determined how well the different features or feature sets handle a reduction of the duty cycle. This relation may be included in a look-up table.

For instance, the look-up table may specify an agreement measure of a feature or feature set at a duty cycle with a physiological state of interest. Possible agreement measures include Cohen's kappa coefficient, an accuracy, a precision, a recall, a true positive rate, a true negative rate. These measures of agreement apply to the determination of the physiological state. The agreement describes an extent to which a determined physiological state based on the feature agrees with the truth (when a certain classifier is used). For instance, in the case of sleep staging, the accuracy may correspond to a percentage of the night that was determined correctly. Cohen's kappa goes further by taking into account “agreement by chance”.

The agreement measures precision, recall, true positive/negative rates can be computed for a specific sleep stage. For instance, in a use-case where the user is only interested in determining the periods of deep sleep, the agreement of the determination of other sleep stages may not be relevant and can thus permit a lower duty cycle as long as precision/recall/etc. for deep sleep are acceptable. As an alternative to making use of a look-up table it may also be possible to make use of directly linear or non-linear connection linking the duty cycle to an estimated significance for a feature (e.g. in the form of a regression). For instance, a relation of a feature to parameters such as an expected root mean square error, an expected absolute error or an expected error percentile level may be exploited.

As explained above, the present invention may be particularly put to use in conjunction with a PPG sensor being duty cycled. However, it is to be understood that also other sensors and other modalities may be used.

For instance, it is known that features derived from a respiration of a subject can also be used for sleep staging, either in combination with cardiac features or alone. Thus, other sensors may be used, such as an accelerometer worn on the area of the thorax configured to measure accelerations in the direction perpendicular to the surface of the thorax. This sensor signal could be used to derive respiration movements which are associated with a respiratory effort of the subject. Furthermore, an inductance plethysmograph embedded in a sleeping shirt tied around the thorax may be used. In addition, it may also be possible to make use of other sensors for measuring a cardiac activity apart from a PPG sensor. Portable or wearable sensors for measuring a heart rate include an accelerometer worn on the area of the thorax configured to measure accelerations in a direction longitudinal to the direction of the body and/or perpendicular to the surface of the thorax (measuring a so-called ballistocardiogram). Also, ECG electrodes embedded on a sleeping shirt tied around the upper body of the subject such that the electrodes are in permanent contact with two points on opposite sides of the chest of the subject may be used.

FIG. 6 schematically illustrates the application of a wearable monitoring apparatus 30 including the monitoring device 16 being applied by a subject 32. The wearable monitoring apparatus 30 is incorporated by a device to be attached to upper arm of the patient, e.g. by means of an arm strap. The apparatus 30 includes a PPG sensor for providing a sensor signal being indicative of a heart rate and a respiratory rate of the subject. Other embodiments of the monitoring apparatus 30 may also have the form of a device for attaching to another limb such as a finger or a wrist etc. The apparatus 30 will usually provide the determined data via an interface such as a display a data connection to the subject and/or to medical support personnel.

One effect of the use of the present invention is that a device or apparatus according to the present invention will usually have varying battery consumption from one use to another.

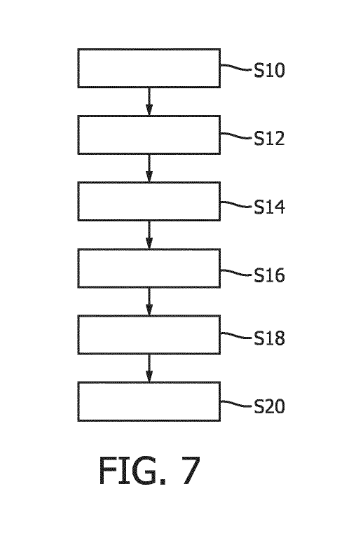

In FIG. 7 a method according to an aspect of the present invention is schematically illustrated. Initially, a sensor signal is obtained (step S10) from a sensor. The sensor signal is indicative of a vital sign of the subject. In particular, a PPG signal is obtained from a PPG sensor included in a wearable device.

In the next step a charge value is obtained (step S12) from a power storage being indicative of the charge state of said power storage. In particular, a battery may represent the power storage.

Based on this charge value the duty cycle of the sensor is controlled (step S14). In particular, the duty cycle is controlled by switching the sensor on or off.

In a next step, this determined duty cycle is analyzed and at least one of the plurality of signal features is selected (S16) based on the duty cycle. In particular, it is advantageous if features are selected that allow providing an accurate and significant indication of the subject's current sleep stage in spite of a reduced duty cycle. For instance, for a reduced duty cycle it may make sense to select another feature than for a signal that is available at a duty cycle of 100%.

In the next step the processing unit is controlled (step S18) to extract this selected at least one feature from the sensor signal.

Then, the selected feature is extracted (step S20) from the sensor signal.

The method of the present invention may, e.g., be carried out by a microprocessor included in a wearable device. The method of the present invention may also be carried out by a server in the internet or by a microprocessor included in a handheld device such as a mobile phone being in communication with a wearable sensor.

In another embodiment, it may also be possible that the duty cycle is not adjusted based on the charge value but that the duty cycle is predefined, e.g. in the form of a sensor-intrinsic function. Then, the duty cycle module will be configured to select a feature set to be extracted and control the processing unit accordingly based on this predefined duty cycle. A corresponding embodiment of the proposed device comprises a sensor interface (18) for obtaining from a sensor (20) a sensor signal indicative of a vital sign of a subject, said sensor being operated at a predefined duty cycle, a processing unit (26) for extracting at least one feature indicative of a physiological state of the subject from the sensor signal, and a duty cycle module (28) for selecting at least one of a plurality of signal features based on the predefined duty cycle and controlling the processing unit (26) to extract said at least one selected signal feature from the sensor signal.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. Device for monitoring a physiological state of a subject comprising:
a sensor interface configured to obtain from a sensor a sensor signal indicative of a vital sign of a subject;
a power storage interface configured to obtain a charge value indicative of a charge state of a power storage powering the sensor;
a duty cycle module configured to
control a duty cycle of the sensor based on the charge value;
select at least one of a plurality of signal features associated with the sensor signal based on the current duty cycle as controlled by the duty cycle module; and control
a processing unit to extract said at least one selected signal feature of the plurality of signal features indicative of a physiological state of the subject from the sensor signal.

2. Device as claimed in claim 1, wherein the processing unit is configured to extract at least one feature indicative of a sleep stage of the subject.

3. Device as claimed in claim 2, wherein the processing unit is configured to determine a hypnogram of the subject indicative of sleep stages of the subject during a predefined time period based on said at least one extracted feature.

4. Device as claimed in claim 3, wherein the processing unit is configured to determine the hypnogram based on a classifier with pre-trained parameters, in particular a Bayesian linear discriminant, trained to assign one of a set of predefined sleep stages to a fraction of said predefined time period.

5. Device as claimed in claim 1, wherein the duty cycle module is configured to reduce the duty cycle if the charge value indicates a charge state below a predefined threshold.

6. Device as claimed in claim 1, wherein the duty cycle module is configured to adjust the duty cycle based on at least one of a required operating time of the sensor, a predicted operating time of the sensor and a required accuracy level of a physiological state monitoring.

7. Device as claimed in claim 1, wherein the sensor interface is configured to obtain at least one of
a sensor signal indicative of a heart rate of the subject, in particular an electrocardiography signal and/or a photoplethysmography signal; and
a sensor signal indicative of a respiration of the subject, in particular an acceleration signal and/or a photoplethysmography signal.

8. Device as claimed in claim 1, wherein the sensor interface is configured to obtain a sensor signal indicative of a heart rate of the subject, in particular a photoplethysmography signal, and the duty cycle module is configured to control the processing unit to extract a feature indicative of a heart rate variability, in particular to extract at least one of:
a mean inter beat interval of the heart rate;
a standard deviation of the mean inter beat interval;
a low frequency power parameter indicative of a power in the spectral band between 0.04 and 0.15 Hz;
a high frequency power parameter indicative of a power in the spectral band between 0.15 and 0.4 Hz;
a mean of absolute successive inter beat intervals differences;
a root-mean-square of successive inter beat intervals differences;
a percentage of successive inter beat interval differences larger than 50 ms;
a standard deviation of successive inter beat interval differences;
a phase of the high frequency pole;
a sample entropy; and
a Teager-Kaiser energy.

9. Device as claimed in claim 1, wherein the duty cycle module is configured to select the at least one of a plurality of signal features based on a predetermined look-up table indicating for a duty cycle a significance of a feature or feature set with respect to a physiological state of the subject.

10. Device as claimed in claim 9, wherein the look-up table indicates a Cohen's kappa coefficient for a duty cycle and a feature or feature set.

11. Wearable monitoring apparatus, comprising:
a device as claimed in claim 1;
a sensor for providing a sensor signal; and
a power storage for powering the sensor and for providing a charge value indicative of a current charge state.

12. Method for monitoring a subject, comprising the steps of:
obtaining from a sensor a sensor signal indicative of a vital sign of a subject;
obtaining a charge value indicative of a charge state of a power storage powering the sensor;
controlling the duty cycle of the sensor based on the charge value;
selecting at least one of a plurality of signal features associated with the sensor signal based on the current duty cycle;
controlling a processing unit to extract said at least one signal feature of the plurality of signal features; and
extracting said at least one signal feature of the plurality of signal features indicative of a physiological state of the subject from the sensor signal.

13. Computer program comprising program code means for causing a computer to carry out the steps of the method as claimed in claim 12 when said computer program is carried out on the computer.
